(19) United States
(12) Patent Application Publication
(10) Pub. No.: US 2026/0114855 A1
(43) Pub. Date: Apr. 30, 2026

(54) ENVIRONMENTALLY SENSITIVE BACTERIA-CAPTURING MICROPARTICLES

(71) Applicant: The Curators of the University of Missouri, Columbia, MO (US)

(72) Inventors: Bret Ulery, Columbia, MO (US); Craig Franklin, Columbia, MO (US); James Amos-Landgraf, Columbia, MO (US); Aaron Ericsson, Columbia, MO (US); Bryce Lindaman, Columbia, MO (US); Dronareddy Madugula, Columbia, MO (US); Nick Bender, Columbia, MO (US); Samantha Huddleston, Columbia, MO (US)

(21) Appl. No.: 19/372,957

(22) Filed: Oct. 29, 2025

Related U.S. Application Data
(60) Provisional application No. 63/713,342, filed on Oct. 29, 2024.

(51) Int. Cl. A61B 10/00; C08G 69/10; C12Q 1/24; C12Q 1/689

(52) U.S. Cl. CPC A61B 10/0045; C08G 69/10; C12Q 1/24; C12Q 1/689; A61B 2010/0061

(57) ABSTRACT

The present disclosure relates to compositions and methods for capturing bacteria at a pH less than 6. The compositions include pH-sensitive polyamide polymer-coated microparticles, wherein the pH-sensitive polyamide polymer is covalently coupled with the microparticle and wherein the pH-sensitive polyamine polymer selectively captures bacteria at a pH less than 6.

### CROSS REFERENCE TO RELATED APPLICATIONS

This application claims the benefit of and priority to U.S. Provisional Patent Application Ser. No. 63/713,342, filed Oct. 29, 2024, the disclosure of which is incorporated by reference in its entirety.

### STATEMENT OF GOVERNMENT SUPPORT

This Invention was made with government support under grant number OD010918 awarded by the National Institutes of Health. The government has certain rights in the invention.

### BACKGROUND OF THE DISCLOSURE

The present disclosure relates generally to research and medicine. More particularly, the present disclosure relates to compositions including a pH-sensitive polymer and methods for using the compositions to capture human and animal gut microbes in the gastrointestinal tract.

The gastrointestinal tract of humans and other vertebrates exhibits regional differences in the resident microbiota. The microbes present perform fundamental biological roles including region-specific metabolism of ingesta, development and tolerance of the immune system, and protection from infectious agents. However, currently, there is a lack of economical noninvasive commercially available systems for surveying the regional gastrointestinal tract microbial contents, and existing alternatives often either involve invasive procedures or expensive engineered devices. Additionally, developing these systems in murine models is prohibitive because of size and physiology. Addressing this gap holds immense potential for reshaping the landscape of human and animal gut microbiome research, facilitating longitudinal studies, and ushering in new frontiers in gastrointestinal medicine with significant implications for diagnostics, preventions, and therapeutics.

### BRIEF DESCRIPTION OF THE DISCLOSURE

The present disclosure is generally related to compositions and methods for capturing bacteria at a pH less than 6. The compositions are particularly suitable in methods for capturing bacteria in the upper gastrointestinal tract of a subject. The compositions are particularly suitable in methods for capturing bacteria in a sample having a pH less than 6.

In one aspect, the present disclosure is directed to a composition for capturing bacteria at a pH less than 6 comprising a microparticle and a pH-sensitive polyamine polymer covalently coupled to the microparticle, wherein the pH-sensitive polyamine polymer selectively captures bacteria at a pH less than 6.

In one aspect, the present disclosure is directed to an in vivo method for capturing bacteria in the upper gastrointestinal tract when present in a subject, the method comprising: administering to a subject a composition comprising a plurality of pH-sensitive polyamide polymer-coated microparticles, wherein the pH-sensitive polyamine polymer selectively captures bacteria at a pH less than 6 to form a bacteria-microparticle complex; and collecting the bacteria-microparticle complex from a sample obtained from the subject.

In one aspect, the present disclosure is directed to a method for capturing bacteria in a sample having a pH less than 6, the method comprising: contacting the sample with a composition comprising a plurality of pH-sensitive polyamide polymer-coated microparticles, wherein the pH-sensitive polyamine polymer selectively captures bacteria at a pH less than 6 to form a bacteria-microparticle complex; and collecting the bacteria-microparticle complex.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The disclosure will be better understood, and features, aspects and advantages other than those set forth above will become apparent when consideration is given to the following detailed description thereof. Such detailed description makes reference to the following drawings, wherein:

FIG. 1 depicts poly(histidine) charge states for the selective capture of gut microbiome by microparticles leveraging the pH sensitivity of poly(histidine).

FIG. 2 depicts histidine monomer synthesis by N-carboxyanhydride (NCA) ring formation and deprotonation.

FIG. 3 depicts poly(histidine) synthesis.

FIG. 4 is an illustration depicting in vitro capture of bacteria at pH 5.5 (step 1) followed by washing at pH 7.5 (step 2) and analysis by 16S rRNA and confocal microscopy.

FIG. 5A is a box plot depicting the DNA yields from an equivalent mass of washed beads, after incubation with mock community standards of nine bacteria (ZymoBIOMICS, D6300) at pH 5.5, at pH 5.5 followed by an increase to pH 7.5, or at pH 7.5. n=6/group: p values based on ANOVA.

FIG. 5B is a stacked bar chart depicting the total read count of nine bacterial species from mock community standards detected on washed beads. The polymer-grafted beads captured all 9 bacterial species from the community standard at pH 5.5 and these bacteria remained captured when the pH was raised to 7.5 and did not bind appreciable bacteria at pH 7.5.

FIG. 6 is an illustration depicting in vivo bacterial capture, collection, separation, and analysis by DNA extraction.

FIGS. 7A and 7B are principal coordinate analysis (PCoA) plots depicting Jaccard dissimilarities between capture beads isolated from feces and different regions of the gastrointestinal tract sampled post-mortem in mice. FIG. 7A depicts bacterial DNA recovered from polymer-grafted microparticles isolated from the feces and different regions of the gastrointestinal tract sampled post-mortem in mice with a low-richness native mouse gut microbiome (GM^{LOW}). FIG. 7B depicts bacterial DNA recovered from polymer-grafted microparticles isolated from the feces and different regions of the gastrointestinal tract sampled post-mortem in mice with a high-richness native mouse gut microbiome (GM^{HIGH}).

FIGS. 8A and 8B are principal coordinate analysis (PCoA) plots depicting Bray-Curtis dissimilarities between capture beads isolated from feces and different regions of the gastrointestinal tract sampled post-mortem in mice. FIG. 8A depicts bacterial DNA recovered from polymer-grafted microparticles isolated from the feces and different regions of the gastrointestinal tract sampled post-mortem in mice with a low-richness synthetic human gut microbiome (hCom1). FIG. 8B depicts bacterial DNA recovered from polymer-grafted microparticles isolated from the feces and different regions of the gastrointestinal tract sampled post-mortem in mice with a high-richness synthetic human gut microbiome (hCom2).

### DETAILED DESCRIPTION

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

The present disclosure is directed to a novel innovative method involving a pH-sensitive polymer that can favorably associate with bacteria in low pH conditions in the upper gastrointestinal tract regions that are present in the stomach, duodenum, and upper jejunum, and resist capture of microbes in the lower GI (e.g., colon) as they travel through the GI system. The compositions and methods use the variation in pH that is known to be more acidic in the upper GI and most of the small intestine before increasing to more neutral pH near the terminal ileum. By linking a polymer that changes binding ability and conformation while transitioning between acidic (permissible capture) and neutral (resistant capture) pH values, an upper GI microbiota capture device has been developed. A series of in vitro, ex vivo, and in vivo experiments indicate a favorable association between the sampling polymer and bacterial cells.

The present disclosure is directed to compositions including a particle and a pH-sensitive polyamide polymer and methods for using the compositions to capture human and animal gut microbes in the gastrointestinal tract. The present disclosure is directed to methods for using the compositions to capture microbes in acid environments.

In one aspect, the present disclosure is directed to a composition for capturing bacteria at a pH less than 6 comprising a microparticle and a pH-sensitive polyamine polymer covalently coupled to the microparticle, wherein the pH-sensitive polyamine polymer selectively captures bacteria at a pH less than 6.

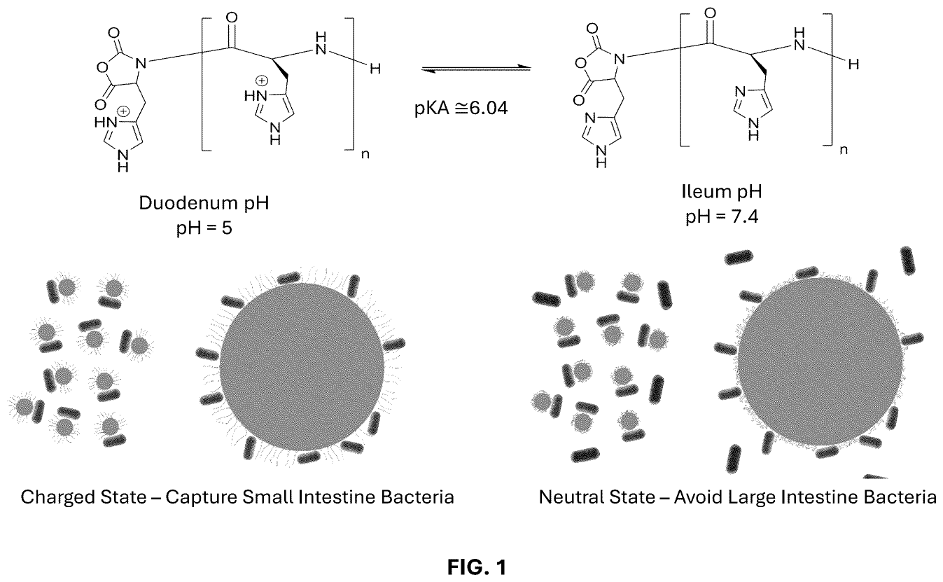

In an exemplary embodiment, the pH-sensitive polyamide polymer includes polyhistidine. Histidine has a side chain pKa value of 6.04. At an acid pH, histidine exists in a charged state where it is available to capture bacteria in the upper gastrointestinal tract where the pH is acidic (See, FIG. 1). As the pH-sensitive polyamide polymer-coated microparticle moves through the gastrointestinal tract and reaches the ileum where the pH increases to neutral and then basic pH, bacteria captured by the polyhistidine polymer in the upper gastrointestinal tract remain in a captured state associated with the pH-sensitive polyamide polymer-coated microparticle. Bacteria existing in the lower gastrointestinal tract are not captured by the polyhistidine polymer.

Microparticles (also interchangeably referred to herein as “beads” and “particles”) that are suitable for use in the composition include magnetic particles, fluorescent particles, polymer microspheres, and combinations thereof. Suitable magnetic particles include superparamagnetic iron oxides (SPIONs), magnetite (Fe₃O₄), maghemite (γ-Fe₂O₃) and ferrites (spinel MFe₂O₄), manganese ferrite (MnFe₂O₄), cobalt ferrite (Co Fe₂O₄), nickel ferrite (Ni Fe₂O₄), zinc ferrite (Zn Fe₂O₄); hematite (α-Fe₂O₃); and particles having metallic and bimetallic cores (Fe, FeCo, and FePt). Suitable fluorescent particles include dyed polystyrene (PS) microspheres, melamine-formaldehyde (MF) fluorescent microspheres, poly(methylmethacrylate) (PMMA) and other polymer microspheres, lanthanide-based and time-resolved particles (e.g., europium/terbium chelate-doped beads), silica particles, quantum dots, carbon dots, and other particle types.

Microparticle diameter can range from nanometers to millimeters including from about 10 nm to about 1 mm, including about 50 nm to about 500 μm (micrometers), including from about 75 nm to about 250 μm. It is desirable to select a microparticle size to avoid internalization by cells, movement through tissues, and/or movement between cells (such as through tight junctions).

It should be understood that different microparticles can be mixed before use. In one example, pH-sensitive polyamide polymer-microparticles prepared using magnetic microparticles can be mixed with pH-sensitive polyamide polymer-microparticles prepared using fluorescent microparticles to create a mixture of magnetic pH-sensitive polyamide polymer-microparticles with fluorescent pH-sensitive polyamide polymer-microparticles.

The polyamide is covalently coupled to a microparticle. In one embodiment, a polyhistidine polymer is covalently coupled to the microparticle using a maleimide-thiol bond.

In some embodiments, the pH-sensitive polyamide polymer further includes glutamic acid, aspartic acid, and combinations thereof. Glutamic acid has a side chain pKa value of 4.25. Aspartic acid has a side chain pKa value of 3.86. This allows for the polymer to possess an overall negative charge at pH >6 assisting polymer-microparticles in repelling bacteria at neutral or under basic conditions.

In some embodiments, the pH-sensitive polyamide polymer further comprises lysine, arginine, and combinations thereof. Lysine has a side chain pKa value of 10.79. Arginine has a side chain pKa value of 12.48. This allow for the polymer to possess a greater positive charge at pH<6 assisting polymer-microparticles in complexing bacteria under acidic conditions.

In an exemplary embodiment, a pH-sensitive polymer is covalently coupled to the microparticle by dissolving Boc-His (Trt)-OH in deuterated dichloromethane (CD₂Cl₂). Thionyl chloride is added to the Boc-His (Trt)-OH THF to initiate an N-carboxyanhydride (NCA) cyclization reaction and precipitated in diethyl ether. The hydrochloride-conjugated NCA His (Trt) monomer is then reacted with triethylamine in EtAc. The resulting NCA His (Trt) solid is filtered, washed, and recrystallized from an EtAc and hexane mixture. His (Trt) NCA is then polymerized in dichloromethane (DCM) using 1-(2-aminoethyl)-maleimide initiator. Microparticles are conjugated with (1-ethyl-3-[3-dimethylaminopropyl]carbodiimide hydrochloride) (EDC) and N-hydroxysuccinimide (NHS) and then with cysteamine to leave a terminal thiol on the particle surface. The maleimide group of the polymer initiator was then conjugated to the thiol present on the particles using click chemistry to produce the polymer grafted beads. Several other approaches that can be leveraged to couple polymers to microparticles include the use of 1-ethyl-3-(3-dimethylaminopropyl) carbodiimide (EDC)/N-hydroxysuccinimide (NHS) chemistry to create an amide bond, azide to alkyne chemistry to create a triazole bond, and tetrazine to trans-cyclooctene to dicyclic ring bonds

In another aspect, the present disclosure is directed to an in vivo method for capturing bacteria in the upper gastrointestinal tract when present in a subject, the method including: administering to a subject a composition comprising a plurality of pH-sensitive polyamide polymer-coated microparticles, wherein the pH-sensitive polyamine polymer selectively captures bacteria at a pH less than 6 to form a bacteria-microparticle complex; and collecting the bacteria-microparticle complex from a sample obtained from the subject.

The pH-sensitive polyamide polymer coated microparticles include a microparticle and a pH-sensitive polyamine polymer covalently coupled to the microparticle.

The pH-sensitive polyamide polymer-coated microparticles include polyhistidine. The polyhistidine ranges from about 1 histidine to about 100 histidines. In some embodiments, the pH-sensitive polyamide polymer further comprises glutamic acid, aspartic acid, and combinations thereof. In some embodiments, the pH-sensitive polyamide polymer further comprises lysine, arginine, and combinations thereof.

Microparticles suitable for use in the composition include magnetic particles, fluorescent particles, polymer microspheres, and combinations thereof. Suitable magnetic particles include superparamagnetic iron oxides (SPIONs), magnetite (Fe₃O₄), maghemite (γ-Fe₂O₃) and ferrites (spinel MFe₂O₄), manganese ferrite (MnFe₂O₄), cobalt ferrite (Co Fe₂O₄), nickel ferrite (Ni Fe₂O₄), zinc ferrite (Zn Fe₂O₄); hematite (α-Fe₂O₃); and particles having metallic and bimetallic cores (Fe, FeCo and FePt). Suitable fluorescent particles include dyed polystyrene (PS) microspheres, melamine-formaldehyde (MF) fluorescent microspheres, poly(methylmethacry late) (PMMA) and other polymer microspheres, lanthanide-based and time-resolved particles (e.g., europium/terbium chelate-doped beads), silica particles, quantum dots, carbon dots, and other particle types.

Microparticle diameter can range from nanometers to millimeters including from about 10 nm to about 1 mm, including about 50 nm to about 500 μm (micrometers), including from about 75 nm to about 250 μm. As discussed herein, it is desirable to select a microparticle size to avoid internalization by cells, movement through tissues, and/or movement between cells (such as through tight junctions).

As used herein, the “upper gastrointestinal tract” includes the stomach, duodenum, jejunum, and ileum. In humans, it is known that the stomach pH ranges from about 1.5 to about 3.5. It is also known that the duodenum pH ranges from about 5.5 to about 6.0. It is also known that the human jejunum pH ranges from about 6.0 to about 7.4. It is also known that the human ileum pH is about 7.4. In mice, it is known that the stomach pH ranges from about 3 to about 4. It is also known that mouse duodenum pH ranges from about 4.5 to about 5. It is also known that mouse jejunum pH ranges from about 4.8 to about 5. It is also known that mouse ileum pH is about 5 to about 8. The method utilizes the change in pH from the stomach to the ileum by using a polymer that changes binding ability and conformation while transitioning between acidic (permissible capture) and neutral (resistant capture) pH values. As illustrated in FIG. 1, the charged state of poly(histidine) in pH of about 5 in the duodenum allows for the capture of bacteria. When the particles complexed with captured bacteria reach the ileum having a pH of about 6 to about 8, the captured bacteria remain bound, but bacteria residing in the ileum are not captured in the lower GI tract when the poly(histidine) is at a neutral state. Additionally, bacteria captured in the upper GI tract remain bound to the particles at the neutral pH of the ileum and pass through lower GI tract of the subject with the feces. Particles are then collected from a fecal sample obtained from the subject. Particles are separated from the fecal sample. This allows for capture of bacteria in the upper GI tract.

Suitable routes to administer the composition include oral, gastric tube, pill form, capsule form, and introduction of the compounds into the stomach or intestine of the subject.

The composition can be formulated as a suspension in a solution. Suitable solutions include water and beverages.

In the method, bacteria, when present, will bind the pH-sensitive polyamide polymer-microparticles at a pH about 6. Suitably, bacteria, when present, will bind the pH-sensitive polyamide polymer-microparticles at a pH less than 6. Bacteria will remain bound to the pH-sensitive polyamide polymer-microparticles at a pH above 6. Bacteria do not bind to the pH-sensitive polyamide polymer-microparticles at a pH greater than 6.

The method includes collecting the pH-sensitive polyamide polymer-microparticles from a sample obtained from the subject. Suitable samples include a fecal sample, an intestinal sample, and combinations thereof. It is particularly suitable to collect the pH-sensitive polyamide polymer-microparticles from a fecal sample obtained from the subject.

The method further includes separating the pH-sensitive polyamide polymer-microparticles from the sample. In embodiments using magnetic particles, the pH-sensitive polyamide polymer-microparticles can be separated from the sample by applying a magnetic force to attract the particles. In embodiments using fluorescent particles, the pH-sensitive polyamide polymer-microparticles can be separated by flow cytometry. It is also suitable to separate the pH-sensitive polyamide polymer-microparticles from the sample using filtration. It is also suitable to separate the pH-sensitive polyamide polymer-microparticles from the sample using centrifugation. Combinations of these separation methods are also suitable.

After collecting pH-sensitive polyamide polymer-microparticles having captured bacteria, bacteria captured by the pH-sensitive polyamide polymer-microparticles are analyzed. Bacteria can be analyzed directly from the pH-sensitive polyamide polymer-microparticles and/or bacteria can be analyzed by separating bacteria from the pH-sensitive polyamide polymer-microparticles.

Any methods can be used to analyze the bacteria. Suitable methods include microscopy, culture of the bacteria, mass spectrometry, nucleic acid amplification, protein analysis, 16S rRNA gene sequencing, genomic sequencing, fluorescent staining, immunoassays, viability assays, and combinations thereof.

After separation of the pH-sensitive polyamide polymer-microparticles from the sample, captured bacteria can be separated from the pH-sensitive polyamide polymer-microparticles. In one embodiment, the bacteria are separated from the particles by contacting the pH-sensitive polyamide polymer-microparticles with a solution having a pH less than 6. Suitably, the particles can be serially contacted with solutions having decreasing pH. Captured bacteria can also be contacted with a lysis solution.

Bacteria that are separated from the pH-sensitive polyamide polymer-microparticles can be cultured. For example, bacteria are separated from the pH-sensitive polyamide polymer-microparticles and grown in culture dishes on solid media, grown in liquid media, and other methods.

The method further includes analyzing the upper GI microbiota. Suitable analytical methods are known in the art and include, for example, nucleic acid isolation and sequencing and isolation and growth of captured microbes.

In one aspect, the present disclosure is directed to a method for capturing bacteria in a sample having a pH less than 6, the method including: contacting the sample with a composition comprising a plurality of pH-sensitive polyamide polymer-coated microparticles, wherein the pH-sensitive polyamine polymer selectively captures bacteria at a pH less than 6 to form a bacteria-microparticle complex; and collecting the bacteria-microparticle complex.

The pH-sensitive polyamide polymer coated microparticles include a microparticle and a pH-sensitive polyamine polymer covalently coupled to the microparticle.

The pH-sensitive polyamide polymer-coated microparticles include polyhistidine. The polyhistidine ranges from about 1 histidine to about 100 histidines. In some embodiments, the pH-sensitive polyamide polymer further comprises glutamic acid, aspartic acid, and combinations thereof. In some embodiments, the pH-sensitive polyamide polymer further comprises lysine, arginine, and combinations thereof.

Microparticles suitable for use in the composition include magnetic particles, fluorescent particles, polymer microspheres, and combinations thereof. Suitable magnetic particles include superparamagnetic iron oxides (SPIONs), magnetite (Fe₃O₄), maghemite (γ-Fe₂O₃) and ferrites (spinel MFe₂O₄), manganese ferrite (MnFe₂O₄), cobalt ferrite (Co Fe₂O₄), nickel ferrite (Ni Fe₂O₄), zinc ferrite (Zn Fe₂O₄); hematite (α-Fe₂O₃); and particles having metallic and bimetallic cores (e.g., Fe, FeCo, and FePt). Suitable fluorescent particles include dyed polystyrene (PS) microspheres, melamine-formaldehyde (MF) fluorescent microspheres, poly(methylmethacrylate) (PMMA) and other polymer microspheres, lanthanide-based and time-resolved particles (e.g., europium/terbium chelate-doped beads), silica particles, quantum dots, carbon dots, and other particle types.

Microparticle diameter can range from nanometers to millimeters including from about 10 nm to about 1 mm, including about 50 nm to about 500 μm (micrometers), including from about 75 nm to about 250 μm. As discussed herein, it is desirable to select a microparticle size to avoid internalization by cells, movement through tissues, and/or movement between cells (such as through tight junctions).

The method further includes separating the pH-sensitive polyamide polymer-microparticles from the sample. In embodiments using magnetic particles, the pH-sensitive polyamide polymer-microparticles can be separated from the sample by applying a magnetic force to attract the particles. In embodiments using fluorescent particles, the pH-sensitive polyamide polymer-microparticles can be separated by flow cytometry. It is also suitable to separate the pH-sensitive polyamide polymer-microparticles from the sample using filtration. It is also suitable to separate the pH-sensitive polyamide polymer-microparticles from the sample using centrifugation. Combinations of these separation methods are also suitable.

After collecting pH-sensitive polyamide polymer-microparticles having captured bacteria, bacteria captured by the pH-sensitive polyamide polymer-microparticles are analyzed as described herein. Bacteria can be analyzed directly from the pH-sensitive polyamide polymer-microparticles and/or bacteria can be analyzed by separating bacteria from the pH-sensitive polyamide polymer-microparticles.

Any methods can be used to analyze the bacteria as described herein. Suitable methods include microscopy, culture of the bacteria, mass spectrometry, nucleic acid amplification, protein analysis, 16S rRNA gene sequencing, genomic sequencing, fluorescent staining, immunoassays, viability assays, and combinations thereof.

After separation of the pH-sensitive polyamide polymer-microparticles from the sample, captured bacteria can be separated from the pH-sensitive polyamide polymer-microparticles. In one embodiment, the bacteria are separated from the particles by contacting the pH-sensitive polyamide polymer-microparticles with a solution having a pH less than 6. Suitably, the particles can be serially contacted with solutions having decreasing pH. Captured bacteria can also be contacted with a lysis solution.

Bacteria that are separated from the pH-sensitive polyamide polymer-microparticles can be cultured. For example, bacteria are separated from the pH-sensitive polyamide polymer-microparticles and grown in culture dishes on solid media, grown in liquid media, and other methods.

### EXAMPLES

### Materials and Methods

### Polymer Synthesis

Boc-His (Trt)-OH (5.6 g) was dried overnight in a vacuum flask. Before use, tetrahydrofuran (THF) and ethyl acetate (EtAc) were dried using molecular sieves. A 10 mg sample of Boc-His (Trt)-OH was dissolved in deuterated dichloromethane (CD₂Cl₂) and analyzed using proton nuclear magnetic resonance (1H NMR) spectroscopy to verify its chemical structure.

The N-carboxyanhydride (NCA) cyclization reaction was initiated by adding thionyl chloride (0.9 mL) to the Boc-His (Trt)-OH THF, 12 w/v % solution under an inert argon atmosphere. After precipitation in diethyl ether, the solid product was isolated via vacuum filtration and dried thoroughly.

The hydrochloride-conjugated NCA His (Trt) monomer was then reacted with triethylamine in EtAc. The resulting NCA His (Trt) solid was filtered, washed, and recrystallized from an EtAc and hexane mixture with a ratio of 1:5.

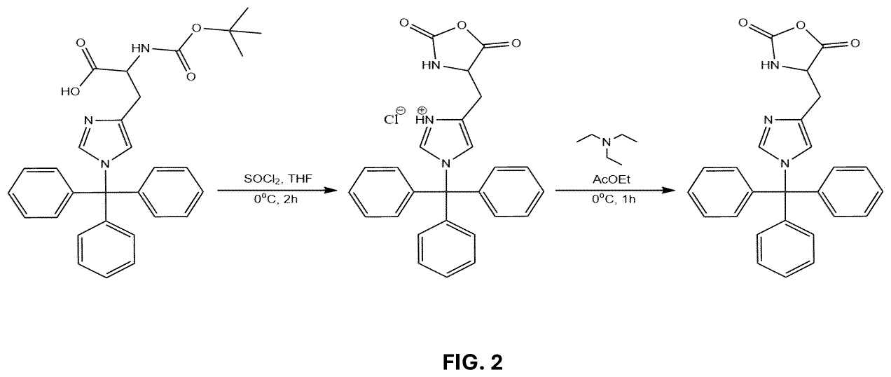

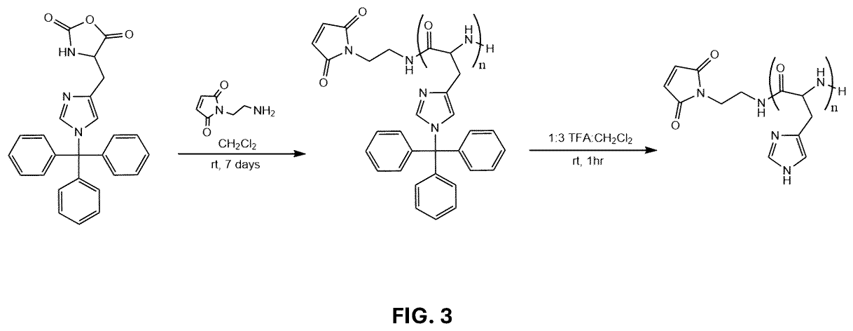

Polymerization of His (Trt) NCA in dichloromethane (DCM) using 1-(2-aminoethyl)-maleimide initiator was carried out over five days, with samples collected daily for analysis via 1H NMR. The final polymer product was obtained by precipitation in diethyl ether, followed by filtration. (Table 1, FIGS. 2 and 3).

TABLE 1
Gel Permeation Chromatogram results of Poly(His)

| M_{w} (g/mol) | Mₙ(g/mol) | PDI | # of Average Monomers |
|---|---|---|---|
| 5,186 | 4,680 | 1.11 | 33 |

### Polymer Grafted Beads Preparation

Carboxylate-terminated ferromagnetic particles with varying sizes, including 105 μm in diameter, were purchased from Spherotech to initiate the project, with subsequent focus on other particle sizes. These were first conjugated with (1-ethyl-3-[3-dimethylaminopropyl]carbodiimide hydrochloride) (EDC) and N-hydroxysuccinimide (NHS) and then with cysteamine to leave a terminal thiol on the microparticle surface, which was verified by Ellman's Reagent. The maleimide group of the polymer initiator was then conjugated to the thiol present on the microparticles using click chemistry. Specifically, 10 mg of microparticles were incubated with the 1.57 mg of the polymer overnight to yield the polymer grafted microparticles.

### In Vitro Bacterial Capture Assay

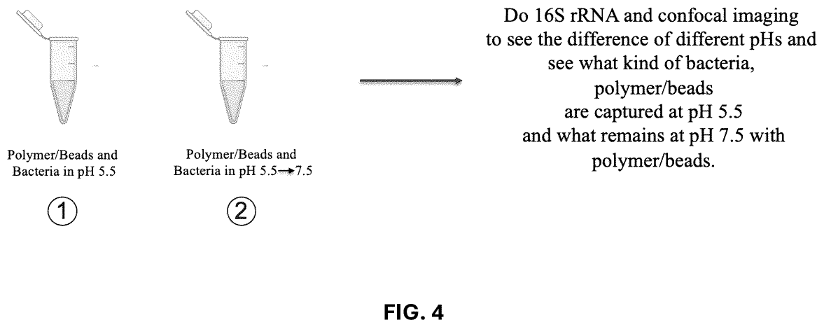

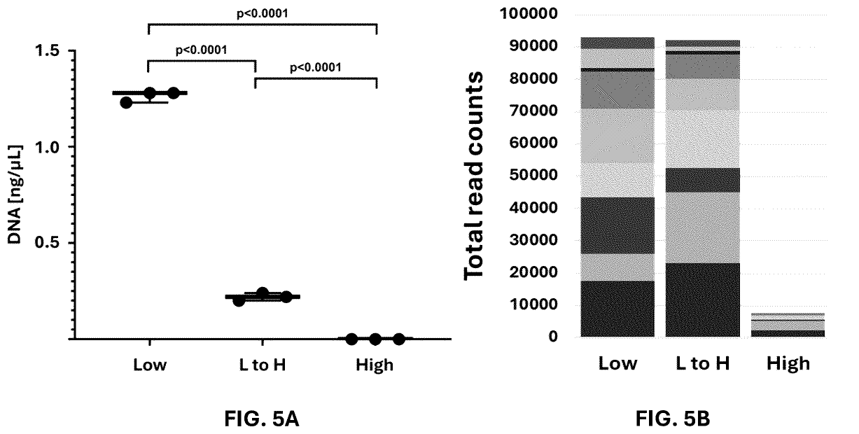

The first experimental trial involved a bacterial sample of Zymogen D6300 Community Standard that has nine known bacterial species in specific quantities. Polymer grafted microparticles were tested for their ability to capture bacteria from this sample (see FIG. 4). Specifically, 100 μL (0.1 w/v %) of polymer grafted microparticles were combined with 50 μL of the bacterial sample at pH 5.5 and incubated for 15-30 minutes (FIG. 4, step 1). The mixture was then washed 5 times with 0.5 mL phosphate-buffered saline (PBS) at pH 7.5 (FIG. 4, step 2). For characterization of bound bacteria, DNA was extracted using Qiagen Powerfecal DNA isolation kit and 16S rRNA analysis was performed to assess relative abundances of bacterial taxa. This experiment was designed to determine whether the polymer grafted microparticles could capture and retain bacteria bound at pH 5.5 and when the pH was raised from 5.5 to 7.5, but resistant to bacteria binding at near neutral pH (7.4). The results of this experiment shows that 1) polymer-grafted microparticles bind bacteria at low pH, is retained when washed at neutral pH, and are resistant to binding when bacteria are introduced to beads at neutral pH polymer-grafted microparticles capture all 9 bacterial species from the community standard at pH 5.5 and these bacteria remain captured when the pH is raised to 7.5, and do not bind appreciable bacteria at pH 7.5 (FIGS. 5A and 5B).

### Ex Vivo Murine Microbiota Capture

All animal work was done in accordance with an approved animal protocol from the Institutional Animal Care and Use Committee at the University of Missouri. To determine if bacterial capture occurs in intestinal contents ex vivo, contents isolated postmortem from mice were used. First, control experiments were performed mixing fecal samples with polymer grafted microparticles or unmodified microparticles to confirm that bacterial capture occurred only when polymer was covalently linked to the ferromagnetic polystyrene microparticles. DNA concentrations were compared to fecal DNA concentrations without microparticle exposure.

### In Vivo Mouse Gavage Experiments

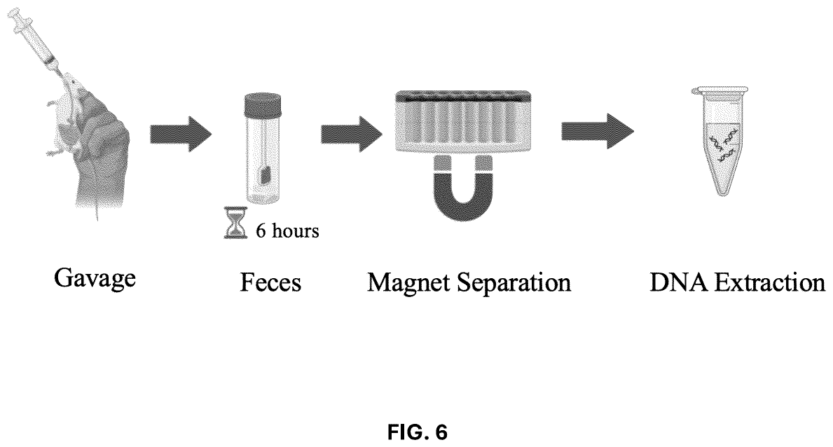

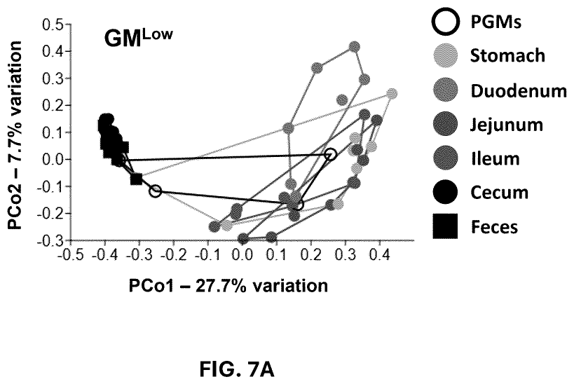

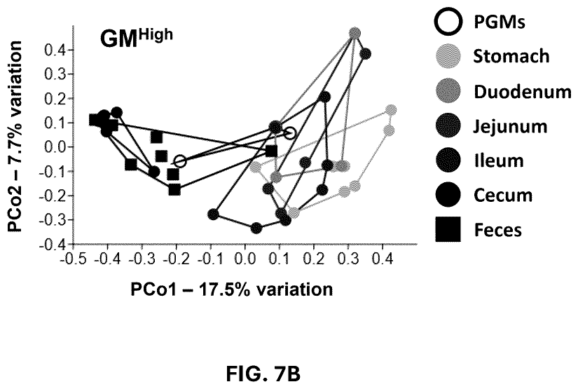

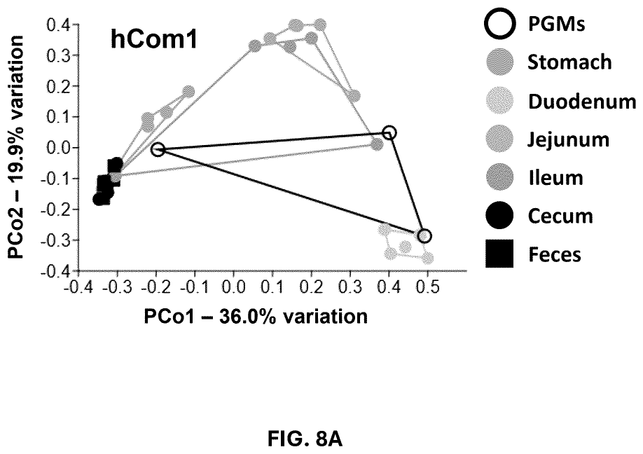

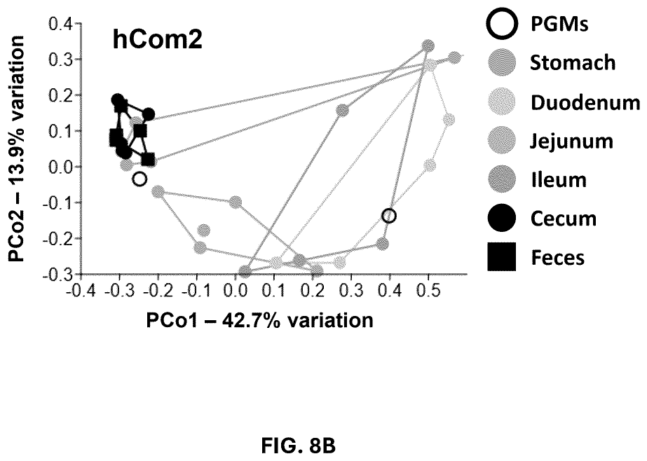

Mice of both sexes were orally administered 200 μL (0.2 w/v %) of polymer grafted ferromagnetic microparticles for the gavage experiment (FIG. 6). Fecal samples were collected at baseline, and at various time points including six hours post-gavage. Necropsy was performed 24 hours post-gavage on all four mice to collect lumenal contents from the stomach, duodenum, jejunum, ileum, cecum, and colon of mice harboring two different native mouse microbiomes (FIG. 7) or two different synthetic human gut microbiomes (FIG. 8). Magnetic separation was performed for all groups followed by 3-5 washing steps and DNA was extracted using the Qiagen Powerfecal kit. The resulting DNA samples were sequenced using a targeted V3-V4 16S rDNA analysis. The transit time in mice is roughly 4-8 hours, which was confirmed from the concentrations of DNA isolated from magnetic microparticles washing of fecal samples.

In vitro experiments include several association and characterization experiments that show that: 1) bacteria specifically binds to polymer-grafted microparticles and not microparticles without polymer: 2) association of microbes occurs more readily in acidic conditions when compared to neutral pH: 3) microbes bound at acidic pH conditions stay associated with the polymer after transitioning to neutral pH; 4) polymer-grafted microparticles bound with microbes at acidic pH conditions in the upper gastrointestinal tract and transitioned to neutral pH conditions in the lower gastrointestinal tract are resistant to adherence of microbes at neutral pH conditions (i.e., successful in vivo capture of upper gastrointestinal tract bacteria); 5) magnetic isolation facilitates purifying the attached microbes from the non-associated microbial populations present at neutral or basic pH. The microbiota associated with the magnetically isolated microparticles can be further characterized using a variety of techniques including but not limited to DNA isolation and sequencing or isolation and growth of captured microbes.

This innovative polymer-based sampling approach enables comprehensive antemortem sampling and analysis of the upper gastrointestinal tract microbiota, which could significantly advance in vivo gut microbiome characterization and targeted therapies and supplements, longitudinal studies, and novel applications in gastrointestinal medicine or environmental sampling under pH-specific conditions.

1. A composition for capturing bacteria at a pH less than 6 comprising:
a microparticle; and
a pH-sensitive polyamine polymer covalently coupled to the microparticle, wherein the pH-sensitive polyamine polymer selectively captures bacteria at a pH less than 6.

2. The composition of claim 1, wherein the pH-sensitive polyamide polymer comprises polyhistidine.

3. The composition of claim 2, wherein the pH-sensitive polyamide polymer further comprises glutamic acid, aspartic acid, and combinations thereof.

4. The composition of claim 2, wherein the pH-sensitive polyamide polymer further comprises lysine, arginine, and combinations thereof.

5. The composition of claim 1, wherein the polyhistidine ranges from about 1 histidine to about 100 histidines.

6. The composition of claim 1, wherein the microparticle is selected from the group consisting of a magnetic bead, a fluorescent particle, a polymer microsphere, a silica particle, a quantum dot, a carbon dot, and combinations thereof.

7. The composition of claim 1, wherein the composition ranges in diameter from about 10 nm to about 1 mm.

8. An in vivo method for capturing bacteria in the upper gastrointestinal tract when present in a subject, the method comprising:
administering to a subject a composition comprising a plurality of pH-sensitive polyamide polymer-coated microparticles, wherein the pH-sensitive polyamine polymer selectively captures bacteria at a pH less than 6 to form a bacteria-microparticle complex; and
collecting the bacteria-microparticle complex from a sample obtained from the subject.

9. The method of claim 8, wherein the plurality of pH-sensitive polyamide polymer-coated microparticles is orally administered to the subject.

10. The method of claim 8, wherein bacteria, when present, will bind the pH-sensitive polyamide polymer-coated microparticles at a pH ranging from about pH 4.0 to less than 6 and will remain bound to the pH-sensitive polyamide polymer-coated particles at pH ranging from about 7.0 to about 9.0.

11. The method of claim 8, further comprising analyzing the bacteria-microparticle complex for the presence of bacteria.

12. The method of claim 8, wherein the plurality of pH-sensitive polyamide polymer-coated particles is collected from a fecal sample obtained from the subject.

13. The method of claim 8, wherein the pH-sensitive polyamide polymer comprises polyhistidine.

14. The method of claim 13, wherein the pH-sensitive polyamide polymer further comprises glutamic acid, aspartic acid, and combinations thereof.

15. The method of claim 13, wherein the pH-sensitive polyamide polymer further comprises lysine, arginine, and combinations thereof.

16. A method for capturing bacteria in a sample having a pH less than 6, the method comprising:
contacting the sample with a composition comprising a plurality of pH-sensitive polyamide polymer-coated microparticles, wherein the pH-sensitive polyamine polymer selectively captures bacteria at a pH less than 6 to form a bacteria-microparticle complex; and
collecting the bacteria-microparticle complex.

17. The method of claim 16, further comprising analyzing the bacteria-microparticle complex for the presence of bacteria.

18. The method of claim 16, wherein the pH-sensitive polyamide polymer comprises polyhistidine.

19. The method of claim 17, wherein the pH-sensitive polyamide polymer further comprises glutamic acid, aspartic acid, and combinations thereof.

20. The method of claim 17, wherein the pH-sensitive polyamide polymer further comprises lysine, arginine, and combinations thereof.
